(19) United States
(12) Patent Application Publication
(10) Pub. No.: US 2024/0302362 A1
(43) Pub. Date: Sep. 12, 2024

(54) TEST SUBSTANCE DETECTION DEVICE AND TEST SUBSTANCE DETECTION METHOD

(71) Applicant: CANON MEDICAL SYSTEMS CORPORATION, Otawara-shi (JP)

(72) Inventor: Hiroto SATAKE, Nasushiobara (JP)

(73) Assignee: CANON MEDICAL SYSTEMS CORPORATION, Otawara-shi (JP)

(21) Appl. No.: 18/595,531

(22) Filed: Mar. 5, 2024

(30) Foreign Application Priority Data
Mar. 8, 2023 (JP) 2023-035408

(51) Int. Cl. G01N 33/543; G01N 15/1434

(52) U.S. Cl. CPC G01N 33/54373; G01N 15/1434; G01N 33/54313

(57) ABSTRACT

A test substance detection device of an embodiment includes a substrate on which a catcher having a binding ability to a test substance is immobilized, an external force applying mechanism, an imaging device, and processing circuitry. The processing circuitry is configured to control the external force applying mechanism to apply an external force to a particle on the substrate, the particle having an ability to bind to the test substance and having undergone a reaction for binding to the test substance, to control the imaging device to image the particle on the substrate in a state in which the external force has been applied, to determine a moving range of the particle due to application of the external force on the basis of the image captured by the imaging unit, and to distinguish between (a) the particle binding to the catcher via the test substance and (b) the particle binding to the catcher without the test substance.

### CROSS-REFERENCE TO RELATED APPLICATION

The present application claims priority based on Japanese Patent Application No. 2023-035408 filed Mar. 8, 2023, the content of which is incorporated herein by reference.

### BACKGROUND OF THE INVENTION

### Field of the Invention

Embodiments disclosed in this specification and drawings relate to a test substance detection device and a test substance detection method.

### Description of Related Art

As a method of detecting a test substance in a sample, a detection technique using a substance that specifically binds to a test substance, such as an antibody, is known. In such a detection technique, it is usually necessary to separate a substance immobilized in a solid phase from a substance that is not immobilized in a solid phase (B/F separation).

On the other hand, a method of measuring the Brownian motion of particles specifically binding to a test substance is known as a technique for detecting a test substance without performing B/F separation. In such a method, a substrate-catcher-test substance-particle complex is formed in a liquid, and the behavior of particles due to the Brownian motion is detected. In a case where a particle is not bonded to the substrate (No Trap), the Brownian motion of the particle is not restricted and thus becomes large. In a case where the particle is non-specifically adsorbed to the substrate without the test substance (Target Negative Trap), the Brownian motion of the particle is restricted and thus becomes almost stationary. In a case where the particle bind via the test substance (Target Positive Trap), the Brownian motion of the particle has an intermediate value.

### BRIEF DESCRIPTION OF THE DRAWINGS

FIG. 1 is a diagram showing an example of a configuration of a test substance detection device of a first embodiment.

FIG. 2 is a schematic diagram showing an example of a configuration of a measuring device in the test substance detection device of the first embodiment.

FIG. 3 is a schematic diagram showing a behavior pattern of particles when an external force in a vertically downward direction is applied to particles on a substrate in the test substance detection device of the first embodiment.

FIG. 4 is a schematic diagram showing a behavior pattern of particles when an external force in a P1 direction within a horizontal plane is applied to particles on a substrate in the test substance detection device of the first embodiment.

FIG. 5 is a schematic diagram showing a behavior pattern of particles when an external force in a P2 direction within the horizontal plane is applied to particles on a substrate in the test substance detection device of the first embodiment.

FIG. 6 is a schematic diagram showing a movable range of particles when an external force in a direction within the horizontal plane is applied to particles on a substrate in the test substance detection device of the first embodiment.

FIG. 7 is a flowchart showing an example of an operation flow of the measuring device in the test substance detection device of the first embodiment.

FIG. 8 is a flowchart showing an example of a processing flow of processing circuitry in the test substance detection device of the first embodiment.

FIG. 9 is a schematic diagram showing a behavior pattern of particles when an external force in a vertically downward direction is applied to particles on a substrate in a test substance detection device of a second embodiment.

FIG. 10 is a schematic diagram showing a behavior pattern of particles when an external force in a vertically upward direction is applied to particles on a substrate in the test substance detection device of the second embodiment.

FIG. 11 is a flowchart showing an example of an operation flow of a measuring device in the test substance detection device of the second embodiment.

### DETAILED DESCRIPTION OF THE INVENTION

Hereinafter, a test substance detection device and a test substance detection method according to embodiments will be described with reference to the drawings. In the drawings, the same or corresponding parts are denoted by the same or corresponding symbols, and redundant description will be omitted. The dimensional ratios in each figure are partially exaggerated for description and do not necessarily match the actual dimensional ratios.

A test substance detection device of an embodiment includes a substrate, an external force applying mechanism, an imaging device, and processing circuitry. A catcher having a binding ability to a test substance is immobilized on the substrate. The processing circuitry controls the external force applying mechanism to apply an external force to a particle, the particle having an ability to bind to the test substance and having undergone a reaction for binding to the test substance. The processing circuitry controls the imaging device to image the particle on the substrate in a state in which the external force has been applied. The processing circuitry determines a moving range of the particle due to application of the external force on the basis of the image captured by the imaging device, and distinguishes between (a) the particle binding to the catcher via the test substance and (b) the particle binding to the catcher without the test substance on the basis of the moving range.

A test substance detection method according to an embodiment includes: (1) reacting a test substance with particle having a binding ability to the test substance on a substrate on which catcher having a binding ability to the test substance is immobilized; (2) applying an external force to the particle on the substrate after (1); (3) imaging the particle on the substrate in a state in which the external force has been applied; (4) determining a moving range of the particle due to application of the external force on the basis of the captured image; and (5) distinguishing between (a) the particle binding to the catcher via the test substance and (b) the particle binding to the catcher without the test substance on the basis of the moving range.

### <Test Substance Detection Device>

### First Embodiment

A test substance detection device of the first embodiment is used to detect a test substance in a sample. In the test substance detection device of the first embodiment, after setting a reference position by applying an external force in a vertically downward direction to particles present on a substrate, an external force is applied in one direction within a horizontal plane. The binding state of the particle to the substrate is determined on the basis of a moving range of the particle from the reference position when an external force in the horizontal direction is applied. In the test substance detection device of the embodiment, the number of particle images captured can be significantly reduced compared to a case where Brownian motion of particle is observed.

A test substance is a substance to be detected, and is not particularly limited. Examples of test substances include peptides, proteins (antibodies, enzymes, hormones, cytokines, etc.), nucleic acids (DNA, RNA, etc.), saccharides (monosaccharides, disaccharides, oligosaccharides, polysaccharides, etc.), physiologically active substances (vitamins, coenzymes, etc.), vesicles (exosomes and other extracellular vesicles, liposomes, etc.), bacteria, cells, viruses, haptens, drugs, drug metabolites, and the like, but are not limited thereto.

A sample used to detect a test substance is not particularly limited. Examples of samples include, for example, body fluids (blood, serum, plasma, lymph, saliva, urine, spinal fluid, nasal discharge, tears, amniotic fluid, tissue fluid), cell extracts, environmental samples (river water, seawater, lake water, groundwater, soil extracts, wastewater), and the like. A sample is prepared as a liquid sample and used in the test substance detection device of the present embodiment.

### [Configuration of Test Substance Detection Device]

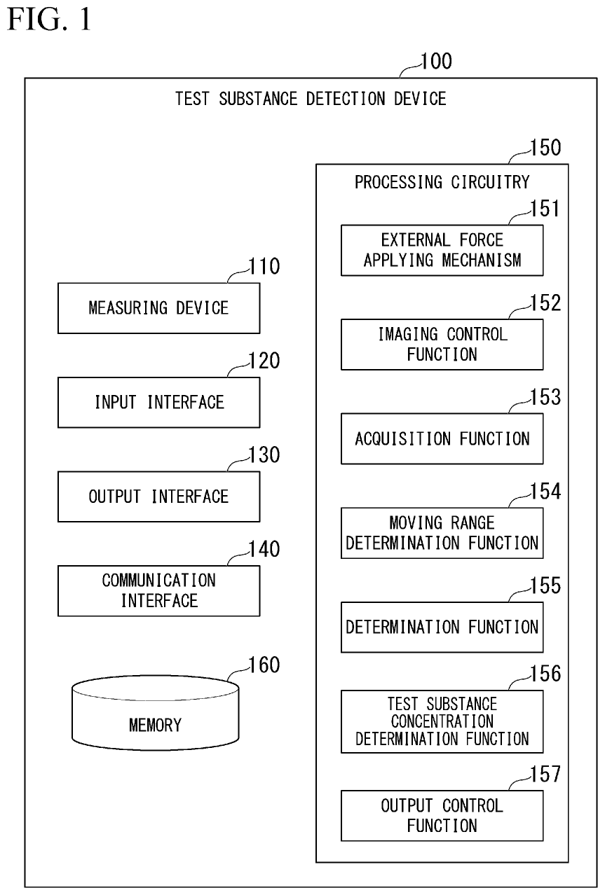

FIG. 1 is a diagram showing an example of a configuration of the test substance detection device 100 of the first embodiment. The test substance detection device 100 includes a measuring device 110, an input interface 120, an output interface 130, a communication interface 140, processing circuitry 150, and a memory 160.

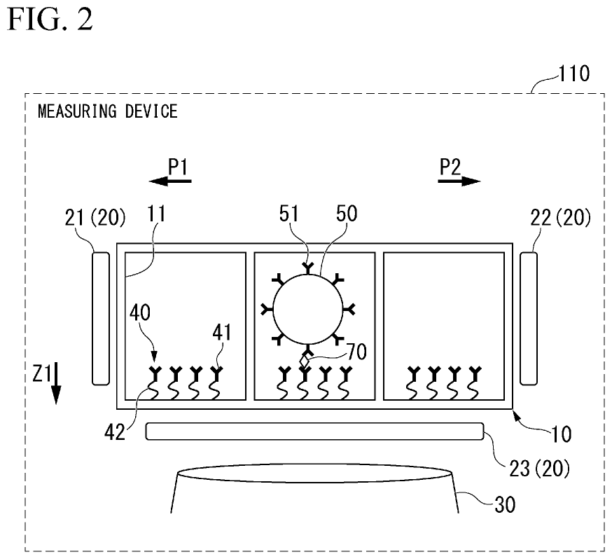

FIG. 2 is a schematic diagram showing an example of a configuration of the measuring device 110. The measuring device 110 includes, for example, a substrate 10, an external force applying mechanism 20, and an imaging device 30. Catchers 40 are immobilized on one surface (upper surface) of the substrate 10. In the measuring device 110, a reaction between the catchers 40, a test substance 70, and particles 50 having a binding ability to the test substance 70 is performed on the substrate 10.

### <<Particles Having Binding Ability to Test Substance>>

The particle 50 is a particle having a binding ability to the test substance 70. An antibody (first antibody 51) having a binding ability to the test substance 70 is immobilized on the particle 50, thereby imparting the binding ability to the test substance 70.

“Having a binding ability to a test substance” means having specific binding activity to a test substance. “Having a specific binding ability” means having high binding affinity for a specific substance, but having only extremely low binding affinity for other substances.

“Antibody” means an immunoglobulin having antigen-binding activity. The antibody does not need to be an intact antibody as long as it has antigen-binding activity, and may be an antigen-binding fragment. As used herein, the term “antibody” encompasses an antigen-binding fragment. An “antigen-binding fragment” is a polypeptide that includes a portion of an antibody and maintains the antigen-binding property of the original antibody. It is preferable that the antigen-binding fragment contain all six complementarity determining regions (CDRs) of the original antibody. That is, it is preferable that the antigen-binding fragment contain all of CDR1, CDR2, and CDR3 of a heavy chain variable region and CDR1, CDR2, and CDR3 of a light chain variable region. Examples of antigen-binding fragments include Fab, Fab′, F(ab′)₂, a variable region fragment (Fv), disulfide bond Fv, single chain Fv (scFv), sc(Fv)₂, and the like.

An antibody may be derived from any organism. Examples of organisms from which antibodies are derived include mammals (humans, mice, rats, rabbits, horses, cows, pigs, monkeys, dogs, etc.), birds (chickens, ostriches), and the like, but are not limited thereto. An antibody may be of any class and subclass of immunoglobulin. An antibody may be a monoclonal antibody or a polyclonal antibody, but a monoclonal antibody is preferred. An antibody can be produced by known methods such as immunization, hybridoma methods, and phage display methods.

The particle 50 is, for example, a magnetic particle. In this case, external force can be applied to the particle 50 using a magnet. Examples of the material of the particle 50 include iron, cobalt, zinc, nickel, oxides, alloys thereof, and the like. A specific example of the particle 50 is a magnetic bead.

Although the shape of the particle 50 is not particularly limited, examples include a sphere, a rectangular parallelepiped, a cube, a triangular pyramid, and shapes close thereto.

The average particle diameter of the particles 50 is preferably 1 μm or less, more preferably 700 nm or less, from the viewpoint of movement according to application of an external force. The average particle diameter of the particles 50 is preferably 1 nm or more, more preferably 10 nm or more, and even more preferably 50 nm or more, from the viewpoint that the particles can be visualized by enlarging them with a microscope or the like. The average particle diameter of the particles 50 is preferably 1 nm to 1 μm, more preferably 10 nm to 1 μm, even more preferably 50 nm to 1 μm, and even more preferably 50 nm to 700 nm. The average particle diameter of the particles 50 is a volume-based median diameter measured by a particle size distribution measuring device using a laser diffraction/scattering method. Examples of such a particle size distribution measuring device include “Microtrac MT3000II” manufactured by Nikkiso Co., Ltd.

A method of immobilizing the first antibody 51 on the particle 50 is not particularly limited. Examples of the immobilization method include a method of using a biotin-avidin bond, a method of using chemical binding between functional groups, and a method of using physical adsorption. Example of the method of using chemical binding include a method of modifying the surface of the particle 50 with a functional group that is reactive with a functional group (carboxy group, amino group, thiol group, or the like) contained in the first antibody 51 and causing the functional groups to react with each other. Examples of combinations of functional groups that perform a binding reaction include a thiol group and a maleimide group, a thiol group and a vinyl group, a hydroxy group and a carboxy group, a hydroxy group and an amino group, a succinimide group and an amino group, and the like. The surface of the particle 50 may be modified with a carboxy group, activated with an active esterifying agent, and then bonded to the amino group of the first antibody 51. Examples of the active esterifying agents include N-hydroxysulfosuccinimide. When a reaction between the functional groups is a condensation reaction, a condensing agent may be used. Examples of the condensing agents include a carbodiimide condensing agent and the like.

The substance that imparts a binding ability to a test substance to particles may not be an antibody. Any substance that has a binding ability to a test substance (also referred to as a “specific binding substance for a test substance”) can impart a binding ability to the test substance to the particle by being immobilized on the particle. A specific binding substance for a test substance can be selected depending on the type of the test substance. For example, examples of combinations of a test substance and a specific binding substance include: a peptide and an antibody; a protein and an antibody; a partial sequence of a nucleic acid and a nucleic acid containing a sequence complementary to the partial sequence; a ligand and a receptor thereof; an enzyme and a substrate thereof; an enzyme and an inhibitor thereof; an enzyme and a cofactor thereof; a sugar chain and lectin; a peptide and an aptamer; a protein and an aptamer; a nucleic acid and an aptamer; a transcription control sequence part of a nucleic acid and a transcription control factor thereof; a compound and a polymer compound having a binding ability to the compound, and the like, but are not limited thereto.

The particles do not need to be magnetic particles. In this case, examples of the material of the particles include: metal particles such as gold, silver, copper, aluminum, manganese, titanium, and oxides thereof; resin particles such as polystyrene and latex; silica particles, and the like. Specific examples include metal beads, resin beads, silica beads, and the like. If the particles are not magnetic particles, an external force can be applied by a Coulomb force, a fluid force, or the like.

The particles may be labeled with a labeling substance. Examples of the labeling substances include fluorescent substances (fluorescein, rhodamine, Texas red, tetramethylrhodamine, carboxyrhodamine, phycoerythrin, 6-FAM (trademark), Cy (registered trademark) 3, Cy (registered trademark) 5, Alexa Fluor (registered trademark) series, etc.), enzymes (e.g., β-galactosidase, alkaline phosphatase, glucose oxidase, peroxidase, polyphenol oxidase, etc.). In a case where a labeling substance is used, a fluorescent substance is preferable. Particles labeled with a fluorescent substance can be observed with a fluorescence microscope.

### <<Substrate>>

The catchers 40 are immobilized to one surface (for example, the upper surface) of the substrate 10. The material of the substrate 10 is not particularly limited as long as it can immobilize the catchers 40. Examples of the material of the substrate 10 include: synthetic resins such as polystyrene, fluororesin, silicone resin, acrylic resin, polyester resin, polyurethane resin, polycarbonate resin, and epoxy resin; glass, and the like. It is preferable that the substrate 10 be made of a transparent material.

A plurality of chambers 11 each of which can accommodate one particle 50 are formed on the surface of the substrate 10 on which the catchers 40 are immobilized. The shape of the chamber 11 is not particularly limited as long as it can maintain a field where the test substance, the particle, and the catchers come into contact with each other. Examples of the shape of the chamber 11 include a cylindrical shape, a hemispherical shape, a polygonal columnar shape, a tubular shape, and the like.

The depth (height) of the chamber 11 is preferably five times or less the particle diameter of the particle 50. The depth of the chamber 11 is, for example, 10 μm or less, preferably 5 μm or less, more preferably 2 μm or less, and even more preferably 1 μm or less. The lower limit of the depth of the chamber 11 is, for example, 0.2 μm or more, 0.4 μm or more, 0.6 μm or more, or 0.8 μm.

The opening area of the chamber 11 only needs to be large enough to accommodate the particle 50, and can be set as appropriate depending on the size of the particle 50. The width of the opening of the chamber 11 can be, for example, approximately 1.05 to 5 times the diameter of the particle 50. Specific examples of the width of the opening of the chamber 11 include, for example, 20 mm or less, 1 mm or less, 50 μm or less, 10 μm or less, and 5 μm or less. Specific examples of the lower limit of the width of the opening of the chamber 11 include, for example, 0.5 μm or more, 1 μm or more, or 2 μm or more.

The chamber may be capable of accommodating a plurality of particles. In this case, the capacity of the chamber is, for example, 50 to 1000 μL, 50 to 800 μL, 50 to 500 μL, 50 to 400 μL, or the like. For example, a microwell plate such as a 96-well microwell plate may be used as the substrate, and wells may be used as the chambers. Alternatively, the chamber may not be present on the surface of the substrate as long as a liquid can be held on the substrate.

### <<Catcher>>

The catchers 40 have a binding ability to the test substance and are immobilized on the substrate 10. The catchers 40 may be immobilized on the bottom surface of the chamber 11 or may be immobilized on the side surface. The catchers 40 capture the test substance 70 present on the substrate 10 and immobilize the test substance 70 on the substrate 10.

The catcher 40 is composed of an antibody 41 (second antibody) and a linker 42. The linker 42 immobilizes the antibody 41 on the substrate 10.

The second antibody 41 is an antibody having a binding ability to the test substance. The second antibody 41 may be the same antibody as the first antibody 51 or may be a different antibody from the first antibody 51 as long as it has a binding ability to the test substance 70. The second antibody 41 may be an antibody that binds to the test substance 70 with a different epitope from that of the first antibody 51, for example.

The second antibody 41 is immobilized on the substrate 10 via the linker 42. Since the second antibody 41 is immobilized on the substrate 10 via the linker 42, the binding state of the particle 50 to the catcher 40 can be easily determined.

Examples of the linker 42 include polymer compounds. Examples of the polymer compounds include synthetic polymer compounds, nucleic acids, lipids, and the like. Examples of the synthetic polymer compounds include polyethylene glycol and the like. Nucleic acids include DNA, RNA, and a mixture of DNA and RNA. The nucleic acids may be artificial nucleic acids such as LNA and PNA. Examples of the lipid include phospholipid polymers such as MPC (2-methacryloyloxyethylphosphorylcholine) polymer.

The length of the linker 42 is not particularly limited and can be set as appropriate depending on the size of the chamber 11. The length of the linker 42 is equal to or less than a length obtained by subtracting the diameter of the particle 50 from the width of the chamber 11. The lower limit of the length of the linker 42 is preferably 15 nm or more, more preferably 30 nm or more, and even more preferably 50 nm or more. The upper limit of the length of the linker 42 is preferably 8/10 or less of the width of the chamber 11, more preferably 5/10 or less, and even more preferably 2/10 or less.

A method of immobilizing the linker 42 on the substrate 10 is not particularly limited. Examples of the immobilization method include a method of using biotin-avidin bond, a method of using chemical bond between functional groups, and a method using physical adsorption. Examples of the method of using chemical bond include a method of modifying the substrate 10 with a specific functional group, labeling the linker 42 with a functional group that reacts with the functional group, and causing the two functional groups to react with each other. Examples of combinations of functional groups that perform the binding reaction include those mentioned above.

Binding of the second antibody 41 and linker 42 can be performed using a known method. Examples of the binding method include a method of using biotin-avidin bond and a method of using chemical bond between functional groups.

The catcher may contain a specific binding substance other than antibodies. A specific binding substance for a test substance can be selected depending on the test substance and examples thereof include the same substances as mentioned above.

### <<External Force Applying Mechanism>>

The external force applying mechanism 20 applies an external force in a predetermined direction to the particles 50 present on the substrate 10. The external force applying mechanism 20 can apply an external force in at least one direction within a horizontal plane to the particles 50 on the substrate 10. It is preferable that the external force applying mechanism 20 be capable of applying an external force in a vertically downward direction to the particles 50 on the substrate 10. As used herein, the “horizontal plane” does not need to be a completely horizontal plane and may also include a plane slightly deviated from the horizontal plane. As used herein, the “vertically downward direction” does not need to be a complete vertically downward direction and may include a direction slightly deviated from the vertically downward direction.

The external force applying mechanism 20 includes, for example, a magnet 21, a magnet 22, and a magnet 23. The magnets 21 and 22 are arranged around the substrate 10 and apply an external force to the particles 50 on the substrate 10 within the horizontal plane. The magnet 23 is arranged below the substrate 10 and applies an external force in a vertically downward direction to the particles on the substrate 10. The magnet 21, the magnet 22, and the magnet 23 are, for example, electromagnets. When the magnet 23 is energized, an external force is applied to the particles 50 in the vertically downward direction (Z1 direction). When the magnet 21 is energized, an external force in a P1 direction within the horizontal plane is applied to the particles 50. When the magnet 22 is energized, an external force in a P2 direction within the horizontal plane is applied to the particles 50. The external force applying mechanism 20 is an example of an “external force applying unit.”

The external force applying mechanism 20 may include an additional magnet which is not illustrated. For example, the number and arrangement of magnets can be set depending on the direction in which an external force is applied. Magnets may be moved depending on the direction in which an external force is applied.

In a case where the magnets 21, 22, and 23 are permanent magnets, the direction of an external force may be controlled by moving these permanent magnets or by moving a shielding plate that shields the magnetic force.

In a case where the particles 50 are not magnetic particles, the external force applying mechanism may apply Coulomb force, fluid force, or the like. For example, in a case where the particles 50 are electrically charged particles, an external force can be applied to the particles by arranging electrodes around the substrate 10 and applying electricity. For example, an external force due to fluid force can be applied to the particles by using a flow cell as a chamber and generating a flow in a liquid in the chamber using a pump or the like.

### <<Imaging Device>>

The imaging device 30 is not particularly limited as long as it can capture still images. Examples of the imaging device 30 include a camera equipped with imaging elements such as CCDs or CMOSs. The imaging device 30 may be connected to an objective lens or an eyepiece of a microscope. The imaging device 30 is an example of an “imaging unit.”

Returning to the description of the configuration diagram of the test substance detection device 100 in FIG. 1, the input interface 120 receives various input operations from the user, converts the received input operations into electrical signals, and outputs the electrical signals to the processing circuitry 150. The input interface 120 includes, for example, physical operation parts such as a mouse, a keyboard, a trackball, a switch, a button, a joystick, and a touch panel. The input interface 120 may be, for example, a user interface that receives voice input, such as a microphone. If the input interface 120 is a touch panel, the input interface 120 may also have a display function such as a display.

The input interface 120 is not limited to those equipped with physical operation parts such as a mouse and a keyboard. For example, electrical signal processing circuitry that receives an electrical signal corresponding to an input operation from an external input apparatus provided separately from the device and outputs this electrical signal to a control circuit is also included in examples of the input interface 120.

The output interface 130 includes, for example, a display, a speaker, and the like. The display displays various types of information. For example, the display displays information output by the processing circuitry 150 as an image, or displays a graphical user interface (GUI) for receiving various input operations from the user. For example, the display is a liquid crystal display (LCD), an organic electroluminescence (EL) display, or the like. The speaker outputs information output by the processing circuitry 150 as sound.

The communication interface 140 includes, for example, a network interface card (NIC), an antenna for wireless communication, and the like. The communication interface 140 communicates with the measuring device 110 connected via a cable or the like, and communicates with an external device via a communication network NW.

The communication network NW may refer to any information communication network using telecommunications technology. For example, the communication network NW includes a local area network (LAN), a wide area network (WAN), the Internet, a telephone communication line network, an optical fiber communication network, a cable communication network, a satellite communication network, and the like.

The external device may be, for example, a computer connected to a LAN. That is, the external device may be a computer within an inspection institution where the test substance detection device 100 is installed. Such a computer is also called a workstation. Further, the external device may be a cloud server connected to a WAN or the Internet.

The communication interface 140 may communicate with the measuring device 110 in a wireless manner such as Wi-Fi.

The processing circuitry 150 includes, for example, an external force application control function 151, an imaging control function 152, an acquisition function 153, a moving range determination function 154, a determination function 155, a test substance concentration determination function 156, and an output control function 157. The processing circuitry 150 realizes these functions by, for example, a hardware processor (computer) executing a program stored in the memory 160 (storage circuit).

The hardware processor in the processing circuitry 150 is, for example, circuitry such as a central processing unit (CPU), a graphics processing unit (GPU), an application specific integrated circuit (ASIC), a programmable logic device (for example, a simple programmable logic device (SPLD) or a complex programmable logic device (CPLD)) or a field programmable gate array (FPGA). The program may be configured to be directly incorporated into the circuit of the hardware processor instead of being stored in the memory 160. In this case, the hardware processor realizes the functions by reading and executing the program incorporated into the circuit. The program may be stored in advance in the memory 160, or may be stored in a non-transitory storage medium such as a DVD or a CD-ROM and installed in the memory 160 from the non-transitory storage medium when the non-transitory storage medium is set in a drive device (not illustrated) of the test substance detection device 100. The hardware processor is not limited to being configured as a single circuit and may be configured as one hardware processor by combining a plurality of independent circuits to realize each function. Further, a plurality of components may be integrated into one hardware processor to realize each function.

The external force application control function 151 controls the external force applying mechanism 20. The external force application control function 151 and the external force applying mechanism 20 are an example of an “external force applying unit.”

The imaging control function 152 controls the imaging device 30. The imaging control function 152 and the imaging device 30 are an example of an “imaging unit.”

The acquisition function 153 acquires an image captured by the imaging device 30, for example, via the communication interface 140.

The moving range determination function 154 determines a moving range of the catchers 40 due to application of an external force on the basis of the image acquired by the acquisition function 153. The moving range determination function 154 is an example of a “determination unit.”

The determination function 155 distinguishes between (a) the particles binding to the catchers through the test substance and (b) the particles binding to the catchers without the test substance on the basis of the moving range of the particles 50 determined by the moving range determination function 154. The determination function 155 is an example of a “determination unit.”

The test substance concentration determination function 156 determines the concentration of the test substance 70 in the sample on the basis of the number or proportion of particles determined by the determination function 155 to be (a) the particle that bind to the catchers via the test substance.

The output control function 157 outputs, via the output interface 130, the image acquired by the acquisition function 153, the moving range of the particles 50 determined by the moving range determination function 154, the determination result of the determination function 155, and the test substance concentration determined by the test substance concentration determination function 156, and the like.

The memory 160 is realized by, for example, a semiconductor memory element such as a random access memory (RAM) or a flash memory, a hard disk, or an optical disc. These non-transitory storage media may be realized by other storage devices connected via the communication network NW, such as a network attached storage (NAS) and an external storage server device. Furthermore, the memory 160 may include a non-transitory storage medium such as a read only memory (ROM) or a register.

### [Method of Determining Binding State of Particles]

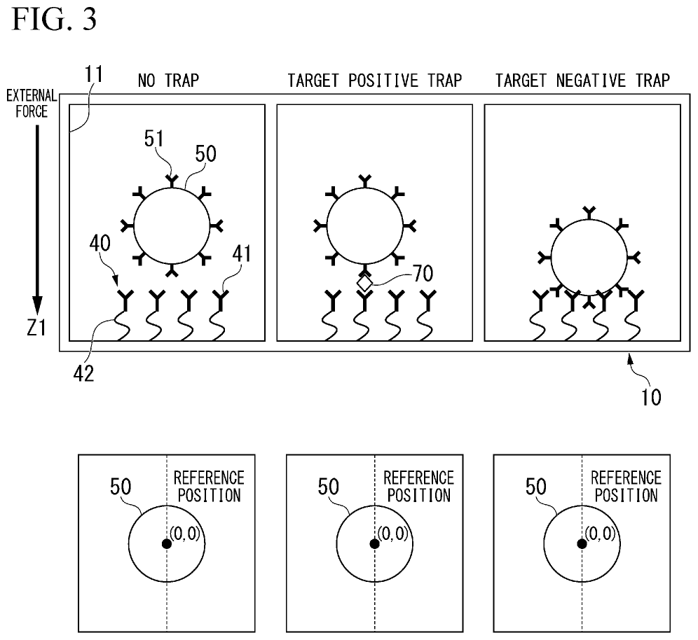

A method of determining a particle binding state by the test substance detection device 100 will be described on the basis of FIGS. 3 to 6.

When the test substance 70 and the particle 50 react with each other on the substrate 10 on which the catchers 40 are immobilized, the test substance 70 binds to the substrate 10. The state of the particle 50 binding to the substrate 10 is typically classified into three patterns. “No Trap” is a state in which the particle 50 does not bind to the substrate 10. “Target Positive Trap” is a state in which the particle 50 binds to the substrate 10 via the test substance 70. “Target Negative Trap” is a state in which the particle 50 non-specifically binds to the substrate 10 without the test substance 70.

The external force applying mechanism 20 applies an external force in the vertically downward direction (Z1 direction) to the particle 50 on the substrate 10 (FIG. 3). The position of the particle 50 at this time is set as a reference position (coordinates (0,0)). The position coordinates of the particle are set, for example, on the basis of the center point of the particle 50 (lower diagram in FIG. 3).

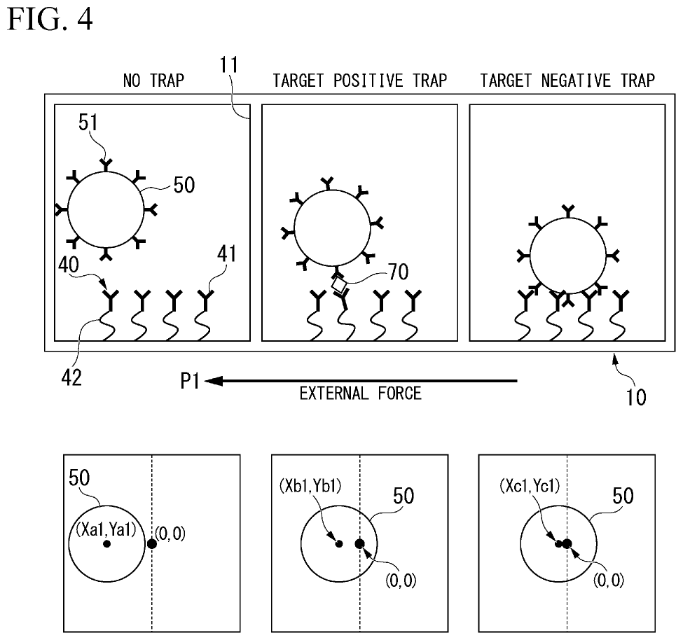

Next, the external force applying mechanism 20 applies an external force in one direction (P1 direction) within the horizontal plane to the particle 50 on the substrate 10 (FIG. 4). At this time, in “No Trap,” the particle 50 is not bound to the substrate 10, and thus the movement of the particle 50 is not restricted. The particle 50 moves largely in the P1 direction. The moving distance (Da1) of the particle 50 from the reference position can be calculated on the basis of the position coordinates (Xa1, Ya1) of the particle 50 at this time (lower diagram in FIG. 4).

In “Target Negative Trap,” the particle 50 is non-specifically trapped by a plurality of catchers 40, and the movement of the particle 50 is greatly restricted. Therefore, the moving distance of the particle 50 is short. The moving distance (Dc1) of the particle 50 from the reference position can be calculated on the basis of the position coordinates (Xc1, Yc1) of the particle 50 at this time (lower diagram in FIG. 4).

In “Target Positive Trap,” the particle 50 is bound to the catcher 40 via the test substance 70. It is conceived that the particle 50 is bound to one catcher 40 via the test substance 70. Although the movement of the particle 50 is restricted by binding to the catcher 40, the degree of restriction is less than that in “Target Negative Trap.” Therefore, the moving distance of the particle 50 falls between those in “No Trap” and “Target Negative Trap.” The moving distance (Db1) of the particle 50 from the reference position can be calculated on the basis of the position coordinates (Xb1, Yb1) of the particle 50 at this time (lower diagram in FIG. 4). That is, the moving distance of the particle 50 is Dc1<Db1<Da1. When the moving distance of each particle 50 when the external force (P1) has been applied is calculated and a moving distance histogram is created, three peaks appear in the distribution. The peaks are estimated to correspond to “No Trap,” “Target Positive Trap,” and “Target Negative Trap” in order from the peak with the largest moving distance. Therefore, particles 50 classified as the intermediate peak can be distinguished as particles corresponding to “Target Positive Trap.” The histogram may be subjected to Gaussian fitting. The moving distance of the particle 50 is an example of a “particle moving range.”

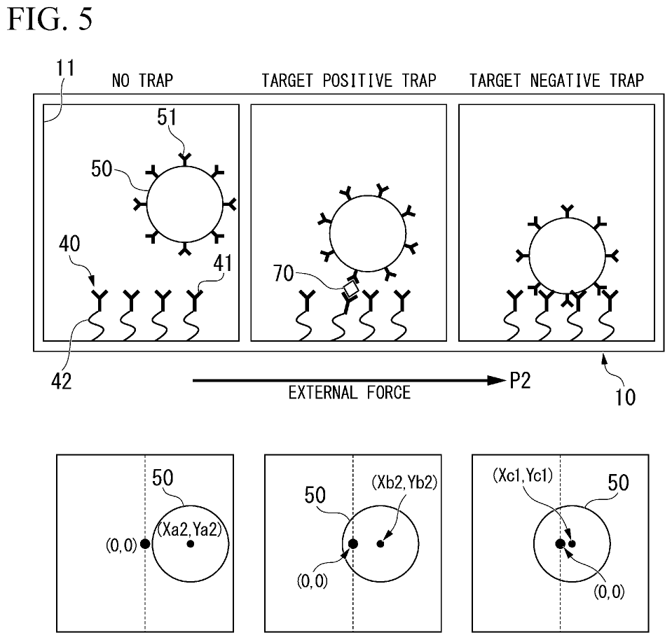

Next, the external force applying mechanism 20 applies an external force in another direction (P2 direction) within the horizontal plane to the particles 50 on the substrate 10 (FIG. 5). At this time, as in the case of FIG. 4, in “No Trap,” the particle 50 moves significantly in the P2 direction. In “Target Negative Trap,” the moving distance of the particle 50 in the P2 direction is short. In “Target Positive Trap,” the moving distance of the particle 50 falls between those in “No Trap” and “Target Negative Trap.” When a moving distance histogram is created for each particle 50, three peaks appear in the distribution. Particles 50 classified as the intermediate peak can be distinguished as particles corresponding to “Target Positive Trap.”

The histogram may be created using the sum of a moving distance when the external force (P1) has been applied and a moving distance when the external force (P2) has been applied. In this case, three peaks also appear in the distribution, and they correspond to “No Trap,” “Target Positive Trap,” and “Target Negative Trap” from the peak with the longest moving distance. Even when external forces are applied in three or more directions within the horizontal plane, the sum of moving distances of the particle 50 when the external forces have been applied in the respective directions may be used to create a histogram. The sum of moving distances of the particle 50 is an example of a “particle moving range.”

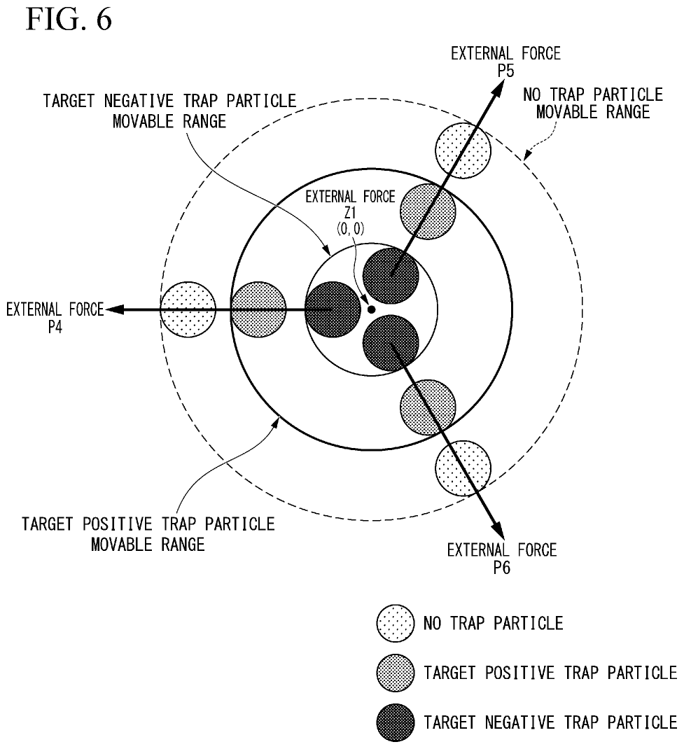

External forces are applied in three or more directions within the horizontal plane, and a movable range of the particle 50 may be estimated from the position coordinates of the particle 50 when the external force has been applied in each direction. The “movable range” of the particle 50 is defined as a spatial area in which the particle 50 can move. An example of an evaluation index for the size of the movable range is the area of the movable range. FIG. 6 is an example of estimating a movable range of the particle 50 from a moving distance of the particle 50 when external forces are applied in three directions (external forces P4 to P6) within the horizontal plane. The position (coordinates (0,0)) of the particle 50 when an external force Z1 in the vertically downward direction has been applied is set as a reference position, and each of the position coordinates (position coordinates (P4), position coordinates (P5), and position coordinates (P6)) of the particle 50 when the external force (P4), external force (P5), and external force (P6) have been applied respectively is acquired. A circle passing through these position coordinates and having the coordinates (0, 0) as a center is set. This circle is estimated to be a movable range of the particle 50. The movable range of the particle 50 can be obtained as the area of this circle. If it is not possible to set a circle having the coordinates (0,0) as a center and passing through the position coordinates (P4), position coordinates (P5), and position coordinates (P6), the area of a polygon formed by connecting the position coordinates (P4), position coordinates (P5), and position coordinates (P6) may be set as the movable range. The movable range of the particle 50 increases in the order of “Target Negative Trap”<“Target Positive Trap”<“No Trap.” When a movable range histogram is created, three peaks appear in the distribution. A particle 50 classified as an intermediate peak can be distinguished as “Target Positive Trap.” The histogram may be subjected to Gaussian fitting. The movable range of the particle 50 is an example of a “particle moving range.”

In a case where the movable range of the particle 50 is estimated by applying external forces in three or more directions within the horizontal plane, setting of a reference position (applying an external force in the vertically downward direction) does not necessarily need to be performed. A circle passing through the position coordinates may be set from each position coordinate of the particle 50 when an external force has been applied in each direction within the horizontal plane without setting a reference position and estimated as the movable range of the particle 50. In a case where an external force is applied in three directions, an intersecting angle of each direction is preferably about 90 to 150 degrees, more preferably about 100 to 140 degrees, even more preferably about 110 to 130 degrees, and especially preferably about 120 degrees.

### [Operation Flow of Test Substance Detection Device]

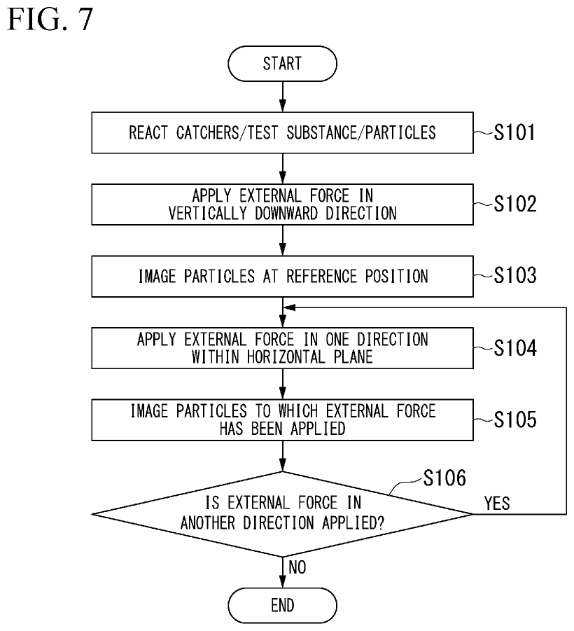

FIG. 7 is a flowchart showing an example of an operation flow of the measuring device 110 in the test substance detection device 100.

In step S101, the catchers 40, the test substance 70, and the particles 50 are reacted on the substrate 10. The reaction is usually carried out in a liquid. Examples of the liquid for the reaction field include a buffer solution. Examples of the buffer solution include a phosphate buffer solution, phosphate buffered saline, a tris buffer solution, a HEPES buffer solution, a borate buffer solution, an acetate buffer solution, a citrate buffer solution, and the like. Examples of reaction temperature include 10 to 60° C., 15 to 50° C., 20 to 50° C., and 30 to 40° C.

A sample containing the test substance 70 and the particles 50 may be simultaneously introduced onto the substrate 10. In this case, the reaction time is not particularly limited, and may be any time that allows the formation of a complex of the catcher 40, the test substance 70, and the particle 50. The reaction time is preferably 15 minutes or more, more preferably 30 minutes or more, and even more preferably 1 hour or more. The upper limit of the reaction time is not particularly limited, but may be 24 hours or less, 12 hours or less, 10 hours or less, 6 hours or less, 4 hours or less, or 2 hours or less, for example.

Alternatively, after the sample containing the test substance 70 is first introduced onto the substrate 10 and a reaction between the test substance 70 and the catchers 40 is performed, the particles 50 are introduced onto the substrate 10 and the test substance 70 and the particles 50 may be reacted. The reaction time in each reaction may be the same as above.

In step S102, the external force applying mechanism 20 applies an external force in the vertically downward direction to the particles 50 on the substrate 10 according to control of the external force application control function 151. Accordingly, a reference position of the particle 50 is set. If the reference position of the particle 50 is not set, step S102 is omitted.

In step S103, the imaging device 30 images the particles 50 at the reference position according to control of the imaging control function 152. Imaging of the particles 50 is performed after the behaviors of the particles 50 have been stabilized in a state in which the external force applying mechanism 20 has applied the external force in the vertically downward direction to the particles 50. If the reference position of the particle 50 is not set, step S103 is omitted.

In step S104, the external force applying mechanism 20 applies an external force in one direction (for example, a first direction) within the horizontal plane to the particles 50 on the substrate 10 according to control of the external force application control function 151. The direction in which the external force is applied is not particularly limited. The magnitude of the external force may be such that binding between the catcher 40 and the test substance 70 and binding between the test substance 70 and the particle 50 do not dissociate. The magnitude of the external force may be varied depending on the properties of the test substance 70, the first antibody 51 immobilized on the particle 50, and the second antibody contained in the catcher 40.

The magnitude of the external force may be determined experimentally, for example, using a purified product of the test substance to be detected. For example, the catchers 40, the purified product, and the particles 50 are reacted, an external force is applied in the vertically downward direction to set a reference position, and an external force is applied in one or more directions within the horizontal plane. The moving range of each particle 50 at this time is determined, and a moving range histogram is created. In the histogram, if three peaks appear in the distribution, these peaks are estimated to correspond to “No Trap,” “Target Positive Trap,” and “Target Positive Trap” in order from the peak with the largest moving range. In this case, the applied external force is determined to have such a magnitude that binding between the catcher 40 and the test substance 70 and binding between the test substance 70 and the particle 50 do not dissociate. Therefore, the magnitude of the external force can be adopted as the magnitude of the external force applied in step S104.

In step S105, the imaging device 30 images the particles 50 according to control of the imaging control function 152. Imaging of the particles 50 is performed after the behaviors of the particles 50 have been stabilized in a state in which the external force applying mechanism 20 has applied an external force in one direction within the horizontal plane to the particles 50.

In step S106, it is determined whether to apply an external force in another direction (for example, a second direction) within the horizontal plane to the particles 50 on the substrate 10. If the determination is “YES,” the operation flow returns to step S104. If the determination is “NO,” the operation flow ends. For example, if there is a direction in which an external force is not applied among directions within the horizontal plane set in advance, the determination is “YES.” If an external force has been applied in all directions in the horizontal plane set in advance, the determination is “NO.”

The number of repetitions of steps S104 to S106 is not particularly limited. The number of repetitions may be, for example, 1 to 10, 1 to 8, 1 to 6, 1 to 5, 1 to 3, or 1 to 2.

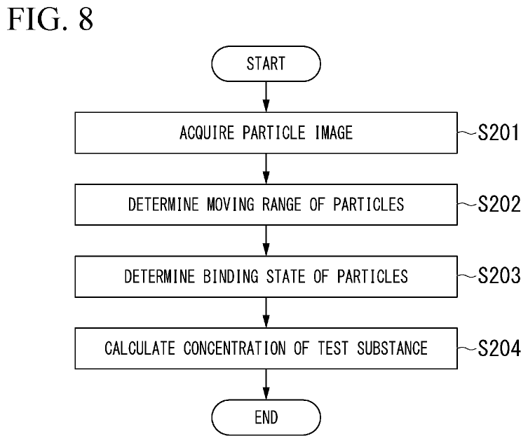

FIG. 8 is a flowchart showing an example of a processing flow of the processing circuitry 150.

In step S201, the acquisition function 153 acquires an image of the particles 50 captured by the imaging device 30 via the communication interface 140 or the like.

In step S202, the moving range determination function 154 determines the moving range of the particle 50 due to application of an external force on the basis of the image of the particle 50 acquired by the acquisition function 153. For example, first, a reference position (coordinates (0, 0)) of the particle 50 is set from an image of the particle 50 captured in a state in which an external force in the vertically downward direction has been applied (refer to FIG. 3). Next, in an image of the particle 50 captured in a state in which an external force has been applied in one direction (for example, the P1 direction) within the horizontal plane, the position coordinates of the particle 50 are determined, and a moving distance from the reference position is calculated (refer to FIG. 4). If there is an image of the particle 50 captured in a state in which an external force has been applied in another direction (for example, the P2 direction) within the horizontal plane, a moving distance of the particle 50 from the reference position is similarly calculated (refer to FIG. 5). The sum of these moving distances may be determined to be a “particle moving range.” Alternatively, the movable range of each particle 50 may be calculated as described with reference to FIG. 6 and determined to be the “particle moving range.”

If the chamber 11 is large enough to accommodate only one particle 50, the same particle 50 between the images can be easily identified on the basis of the position of the chamber 11. That is, the particles 50 located in the same chamber 11 between the images can be identified as the same particle 50.

If the chamber 11 is large enough to accommodate two or more particles 50 or if the chamber 11 is not present, an image when an external force has been applied in the vertically downward direction and an image when an external force has been applied in one direction within the horizontal plane are compared, and the particles 50 that are located close to each other in both images can be determined to be the same particle 50. In this case, “No Trap” particles 50 move a long distance when an external force has been applied in one direction within the horizontal plane and are not present at a position close to the particle 50 at the reference position. Therefore, the particles 50 that can be associated between the images may be determined to correspond to “Target Positive Trap” and “Target Negative Trap,” and the moving ranges of these particles 50 may be determined.

In step S203, the determination function 155 distinguishes each particle 50 and determines the binding state of the particle 50 on the basis of the moving range determined by the moving range determination function 154. That is, the determination function 155 distinguishes among (a) particles 50 bound to the catchers 40 via the test substance 70 (Target Positive Trap), (b) particles 50 bound to the catchers 40 without the test substance 70 (Target Negative Trap), and (c) particles 50 not bound to the catchers 40 (No Trap). For example, when a histogram of moving ranges determined for each particle 50, three peaks appear in the distribution. “No Trap,” “Target Positive Trap,” and “Target Negative Trap” are distributed in order from the peak with the largest moving range. Therefore, the particles 50 classified as the intermediate peak are distinguished as “Target Positive Trap” and the particles 50 classified as the peak with the smallest moving range are distinguished as “Target Negative Trap.”

If the chamber 11 is large enough to accommodate two or more particles 50 or if the chamber 11 is not present, the moving range of “No Trap” cannot be determined in step S202. Therefore, when a moving range histogram is created, there are two peaks. In this case, the particles 50 classified as a peak with a large moving range are distinguished as particles corresponding to “Target Positive Trap” and the particles 50 classified as a peak with a short moving distance are distinguished as particles corresponding to “Target Negative Trap.”

In step S204, the test substance concentration determination function 156 detects the test substance 70 on the basis of the particles 50 distinguished as “Target Positive Trap” and determines the concentration of the test substance 70 in the sample. For example, the particles 50 classified as “Target Positive Trap” are counted, and the concentration of the test substance 70 in the sample is determined on the basis of a calibration curve stored in the memory 160 or the like. Alternatively, the proportion of “Target Positive Trap” in the total number of particles 50 distinguished as “Target Positive Trap” and “Target Negative Trap” may be calculated and the concentration of the test substance 70 in the sample may be determined on the basis of the calibration 5 curve stored in the memory 160 or the like. Accordingly, processing ends.

According to the first embodiment described above, if there are at least two images of an image obtained when an external force has been applied to the particles 50 in the vertically downward direction and an image obtained when an external force has been applied in one direction within the horizontal plane, it is possible to distinguish the particle 50 that is “Target Positive Trap” from the particles 50. Therefore, compared to a case of observing the Brownian motion of the particles 50, the number of images to be captured can be reduced.

### Second Embodiment

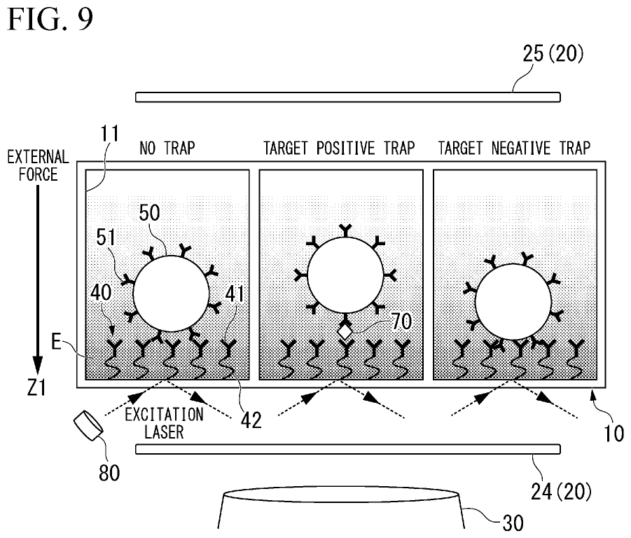

A test substance detection device of the second embodiment includes a light source 80 that irradiates the substrate 10 with an excitation laser at a total reflection angle (refer to FIG. 9). The light source 80 irradiates the substrate 10 with an excitation laser at a total reflection angle to generate evanescent waves. Scattered light from the particle 50 caused by the evanescent waves is attenuated depending on the distance of the particle 50 from the substrate 10. Therefore, the scattered light from the particle 50 is detected and used as an index of the distance of the particle 50 from the substrate 10.

In the second embodiment, as particles 50, for example, magnetic particles labeled with a fluorescent dye may be used. By using the fluorescently labeled particles 50, scattered light caused by evanescent waves can be detected as fluorescence.

The light source 80 is not particularly limited as long as it can emit an excitation laser. Examples of the light source 80 include solid lasers such as YAG laser, ruby laser, glass laser, YVO4 laser, LD laser, and fiber laser, semiconductor lasers, and the like. The light source 80 irradiates the substrate 10 with an excitation laser at a total reflection angle from the opposite side (the lower surface side of the substrate 10) to the surface (the surface on which the chamber 11 is formed) of the substrate 10 on which the catchers 40 are immobilized.

The external force applying mechanism 20 can apply an external force in the vertically downward direction and an external force in a vertically upward direction to the particles 50 on the substrate 10. As used herein, the “vertically upward direction” does not necessarily have to be a completely vertically upward direction and may include directions slightly deviated from the vertically upward direction.

The external force applying mechanism is not particularly limited as long as it can apply an external force in the vertically downward direction and vertically upward direction. The external force applying mechanism 20 includes, for example, a magnet 25 and a magnet 26 arranged above and below the substrate 10. If the external force is Coulomb force, the external force applying mechanism may include, for example, electrodes disposed above and below the substrate 10. If the external force is a fluid force, the external force applying mechanism may include a pump or the like that can generate a flow of liquid on the substrate in the vertically downward direction and the vertically upward direction.

The imaging device 30 is not particularly limited as long as it can image scattered light from the particles due to evanescent wave illumination. An example of the imaging device 30 is a camera connected to an objective lens of a dark field microscope.

The other components can be the same as those of the first embodiment.

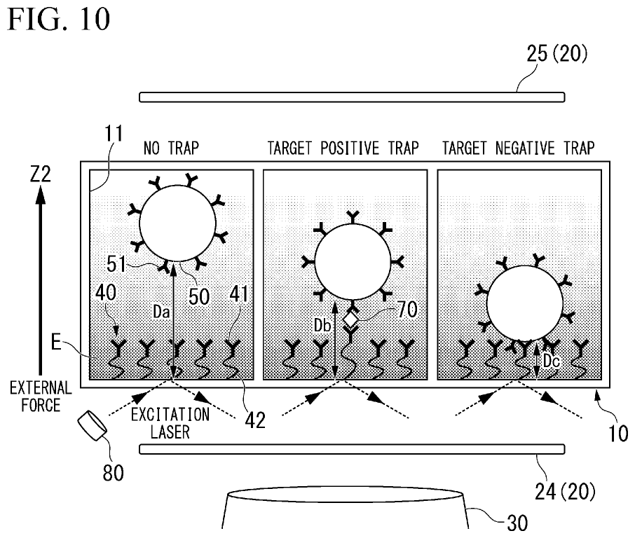

A method of determining the binding state of the particle in the test substance detection device of the second embodiment will be described on the basis of FIGS. 9 and 10.

The light source 80 irradiates the substrate 10 with an excitation laser from below the substrate 10 at a total reflection angle. As a result, evanescent waves E are generated in the space above the substrate 10. The evanescent waves E are attenuated exponentially as the distance from the substrate 10 increases. Scattered light generated from the particle 50 by the evanescent waves E is attenuated as the distance of the particle 50 from the substrate 10 increases. Therefore, the intensity of the scattered light caused by the evanescent waves E is used as an index for the distance of the particle 50 from the substrate 10.

First, the external force applying mechanism 20 applies an external force in the vertically downward direction (Z1 direction) to the particles 50 on the substrate 10 (FIG. 9). In this state, the light source 80 irradiates the substrate 10 with an excitation laser at a total reflection angle. As a result, evanescent waves E are generated on the substrate 10. The intensity of scattered light generated from the particle 50 at this time is set as a reference scattered light intensity (L0).

Next, the external force applying mechanism 20 applies an external force in the vertically upward direction (Z2 direction) to the particles 50 on the substrate 10 (FIG. 10). As a result, the particles 50 move vertically upward. In this state, when the light source 80 irradiates the substrate 10 with an excitation laser at a total reflection angle, evanescent waves E are generated on the substrate 10 and scattered light is generated from the particle 50. The intensity (L1) of this scattered light decreases as the distance of the particle 50 from the substrate 10 increase.

In “No Trap,” the particle 50 is not bound to the substrate 10 and thus the movement of the particle 50 is not restricted. Therefore, when an external force in the vertically upward direction (Z2 direction) is applied, the particle 50 significantly moves upward and the distance (Da) between the substrate 10 and the particle 50 increases. The intensity (L1a) of the scattered light generated from the particle 50 is significantly attenuated compared to the reference scattered light intensity (L0).

In “Target Negative Trap,” the particle 50 is non-specifically trapped by a plurality of catchers 40. Therefore, the movement of the particle 50 is significantly restricted, and thus the movement of the particle 50 is insignificant when an external force in the vertically upward direction (Z2 direction) has been applied. The distance (Dc) between the substrate 10 and the particle 50 does not change much from the reference position. The intensity (L1c) of the scattered light generated from the particle 50 caused by the evanescent waves E is not attenuated much compared to the reference scattered light intensity (L0).

In “Target Positive Trap,” the particle 50 is bound to the catcher 40 via the test substance 70. The movement of the particle 50 is limited by the length of the catcher 40 and the size of the test substance 70. When an external force in the vertically upward direction (Z2 direction) is applied, the mobility of the particle 50 falls between “No Trap” and “Target Negative Trap.” The distance (Db) between the substrate 10 and the particle 50 is slightly greater than the reference position. The intensity (L1b) of the scattered light generated from the particle 50 caused by the evanescent waves E is attenuated compared to the reference scattered light intensity (L0), but the degree of attenuation falls between “No Trap” and “Target Negative Trap.”

For each particle 50, when the difference (L0-L1) between the reference scattered light intensity (L0) and the scattered light intensity (L1) when an external force in the vertically upward direction has been applied is calculated and a histogram is created, three peaks appear in the distribution. The peaks are estimated to correspond to “No Trap,” “Target Positive Trap,” and “Target Negative Trap” in order from the peak with the largest scattered light intensity difference (L0-L1). Therefore, particles 50 classified as the intermediate peak can be distinguished as particles corresponding to “Target Positive Trap.” The histogram may be subjected to Gaussian fitting. The moving distance of the particle 50 using the scattered light intensity difference (L0-L1) as an index is an example of a “particle moving range.”

### [Operation Flow of Test Substance Detection Device]

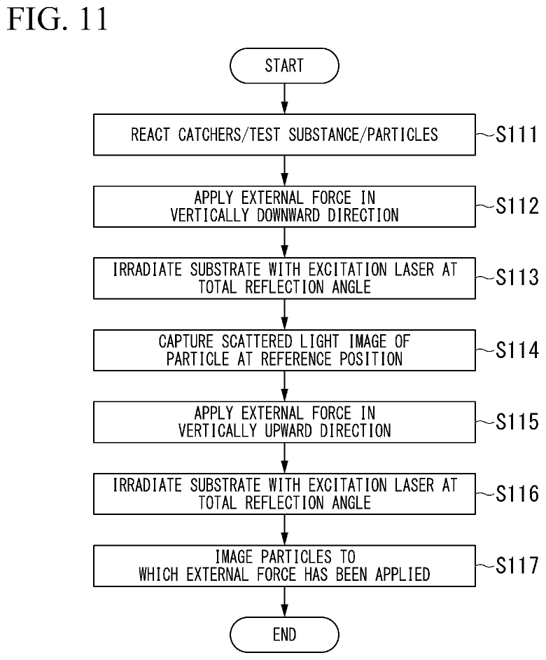

FIG. 11 is a flowchart showing an example of an operation flow of the measuring device in the test substance detection device of the second embodiment.

In step S111, the catchers 40, the test substance 70, and the particles 50 are reacted on the substrate 10. The reaction can be performed in the same manner as in step S101 in the first embodiment.

In step S112, the external force applying mechanism 20 applies an external force in the vertically downward direction to the particles 50 on the substrate 10 according to control of the external force application control function 151. Accordingly, a reference position of the particle 50 is set.

In step S113, the light source 80 irradiates the substrate 10 with an excitation laser from below the substrate 10 at a total reflection angle. Accordingly, evanescent waves E are generated on the substrate 10.

In step S114, the imaging device 30 captures a scattered light image of the particles 50 at the reference position according to control of the imaging control function 152. Imaging of the particles 50 is performed after the behaviors of the particles 50 have been stabilized in a state in which the external force applying mechanism 20 has applied the external force in the vertically downward direction to the particles 50.

In step S115, the external force applying mechanism 20 applies an external force in the vertically upward direction to the particles 50 on the substrate 10 according to control of the external force application control function 151. The magnitude of the external force may be such that binding between the catcher 40 and the test substance 70 and binding between the test substance 70 and the particle 50 are not dissociated. As described above in the first embodiment, the magnitude of the external force may be determined experimentally, for example, using a purified product of the test substance to be detected.

In step S116, the light source 80 irradiates the substrate 10 with an excitation laser from below the substrate 10 at a total reflection angle. Accordingly, evanescent waves E are generated on the substrate 10.

In step S117, the imaging device 30 captures a scattered light image of the particles 50 according to control of the imaging control function 152. Imaging of the particles 50 is performed after the behaviors of the particles 50 have been stabilized in a state in which the external force applying mechanism 20 has applied an external force in one direction within the horizontal plane to the particles 50. Accordingly, a series of operations in the measuring device 110 ends.

The processing flow of the processing circuitry 150 can be performed according to the flowchart in FIG. 8.

In step S201, the acquisition function 153 acquires a scattered light image of the particles 50 captured by the imaging device 30 via the communication interface 140 or the like.

In step S202, the moving range determination function 154 determines the moving range of the particle 50 due to application of the external force on the basis of the image of the particle 50 acquired by the acquisition function 153. In determining the moving range, first, a scattered light intensity (reference scattered light intensity (L0)) is obtained from a scattered light image of the particle 50 which has been captured in a state in which an external force in the vertically downward direction has been applied. Next, a scattered light intensity (L1) is obtained from a scattered light image of the particle 50 which has been captured in a state in which an external force has been applied in the vertically upward direction. The scattered light intensity can be obtained as, for example, fluorescence brightness. Next, the difference (L0-L1) between the reference scattered light intensity (L0) and the scattered light intensity (L1) is obtained. This scattered light intensity difference (L0-L1) serves as an index of the moving range of the particle 50 and thus is determined to be a “particle moving range.”

The same particle 50 between the images can be easily identified on the basis of the positions of the particles 50. That is, the particles 50 located at the same position between the images can be identified as the same particle 50.

In step S203, the determination function 155 distinguishes each particle 50 on the basis of the moving range determined by the moving range determination function 154, and determines the binding state of the particle 50. That is, the determination function 155 distinguishes among (a) particles 50 bound to the catchers 40 via the test substance 70 (Target Positive Trap), (b) particles 50 bound to the catchers 40 without the test substance 70 (Target Negative Trap), and (c) particles 50 not bound to the catchers 40 (No Trap). For example, when a moving range histogram is created for each particle 50, three peaks appear in the distribution. “No Trap,” “Target Positive Trap,” and “Target Negative Trap” are distributed in order from the peak with the largest moving range. Therefore, the particles 50 classified as the intermediate peak are distinguished as particles corresponding to “Target Positive Trap” and the particles 50 classified as the peak with the smallest moving range are distinguished as particles corresponding to “Target Negative Trap.” Here, the scattered light intensity difference (L0-L1) may be 5 used as the moving range of the particle 50 as it is.

In step S204, the test substance concentration determination function 156 detects the test substance 70 on the basis of the particles 50 distinguished as particles corresponding to “Target Positive Trap” and determines the concentration of the test substance 70 in the sample. Accordingly, processing ends.

According to the second embodiment described above, it is possible to distinguish particles corresponding to “Target Positive Trap” from among particles on the basis of two scattered light images: a scattered light image obtained when an external force has been applied in the vertically downward direction to the particles; and a scattered light image obtained when an external force has been applied in the vertically upward direction to the particles. Therefore, compared to a case of observing the Brownian motion of particles, the number of images to be captured can be reduced.

### Other Embodiments

The test substance detection device of the embodiments may include a flow channel structure on the substrate for introducing a sample containing a test substance, a particle suspension, and the like.

The test substance detection device of the embodiments may include a temperature control mechanism for controlling the temperature of a liquid introduced onto the substrate. Examples of the temperature control mechanism include a heater, a cooler, a thermostat, and the like.

The test substance detection device of the embodiments may include a microscope for observing particles on the substrate. The imaging device may be connected to the eyepiece or objective of the microscope.

### <Test Substance Detection Method>

### Third Embodiment

In a test substance detection method of the third embodiment, in a first process, catchers, the test substance, and particles are reacted on a substrate. Accordingly, (a) particles bound to the catchers through the test substance, (b) particles bound to the catchers without the test substance, and (c) particles t not bound to the substrate are present on the substrate. In a second process, an external force in at least one direction within the horizontal plane is applied to the particles on the substrate. In a third process, the particles to which the external force in the direction within the horizontal plane has been applied are imaged. In a fourth process, the moving range of the particle due to application of the external force in the direction within the horizontal plane is determined on the basis of the captured image. In a fifth process, the particles (a) and (b) are distinguished on the basis of the moving range.

### [First Process]

The first process can be performed in the same manner as in step S101 described above. The sample containing the test substance may be diluted or concentrated as appropriate before being introduced onto the substrate 10. For example, if the sample is expected to have a high concentration of the test substance, it may be diluted using a buffer solution or the like. If the sample is expected to have a low centration of the test substance, the test substance may be concentrated using a column or the like.

In the first process, it is desirable from the quantitative viewpoint to react the test substance and the particles in such a manner that “some particles bind to the test substance to form test substance-bound particles and at least one statistically significant particle does not bind to the test substance.” Specifically, when the test substance in the sample is brought into contact with 10⁶ particles having a binding ability to the test substance, for example, the test substrate and the particles are reacted in such a manner that the average number of the test substance molecules on the particle (λ), obtained using the following expression, satisfies λ<14.

λ=[concentration of complex of test substance and antibody]/[concentration of particles]

It is possible to adjust λ depending on the conditions under which the sample containing the test substance is brought into contact with the particles, and λ can be obtained by the concentration of the test substance in the sample, the amount of sample, and the number of particles to be reacted. For example, if the number of the particles is 10⁶, by causing the test substance to react with the particles such that λ<14, at least one statistically significant particle does not bind to the test substance.

The substrate may be blocked before the sample and the particles are introduced. By blocking the substrate, non-specific binding can be reduced. Blocking can be performed using a known blocking solution. Examples of blocking solutions include a buffer solution containing about 1 to 5% skim milk or bovine serum albumin (BSA), and the like. Examples of buffer solutions for the blocking solution include buffer solutions such as a phosphate buffer solution, PBS, a Tris buffer solution, and a HEPES buffer solution, buffer solutions obtained by adding a surfactant such as Tween 20 to the aforementioned buffer solutions, and the like, but are not limited thereto. In the test substance detection method of the embodiment, it is possible to distinguish between particles that are specifically bound to the test substance and particles that are non-specifically bound to the catcher, and thus blocking may not be performed.

In a case where (i) reaction between the catchers immobilized on the substrate and the test substance and (ii) reaction between the test substance and the particles are performed separately, the substrate may be washed after the reaction of (i). As a washing solution, for example, a buffer solution as described above can be used. By performing washing, impurities contained in the sample can be removed from the substrate and non-specific binding can be reduced. In the test substance detection method of the embodiment, it is possible to distinguish between the particles that are specifically to the test substance and the particles that are non-specifically bound to the catcher, and thus washing may not be performed.

### [Second Process]

The second process can be performed in the same manner as in step S104 described above. The direction in which an external force is applied within the horizontal plane may be one or more directions, preferably two or more directions, and more preferably three or more directions. The second process may include applying an external force in the vertically downward direction to the particles on the substrate. Application of an external force in the vertically downward direction can be performed in the same manner as in step S102 described above.

### [Third Process]

The third process can be performed in the same manner as in step S105 described above. In a case where an external force has been applied in a plurality of directions within the horizontal plane, imaging is performed for each direction of the external force. The third process may include imaging the particles to which an external force in the vertically downward direction has been applied. Imaging of the particles to which an external force in the vertically downward direction has been applied can be performed in the same manner as in step S103 described above.

### [Fourth Process]

The fourth process can be performed in the same manner as in step 202 in the test substance detection device of the first embodiment described above.

### [Fifth Process]

The fifth process can be performed in the same manner as in step 203 in the test substance detection device of the first embodiment described above.

### Fourth Embodiment

In a test substance detection method of the fourth embodiment, the second process includes applying an external force in the vertically downward direction and applying an external force in the vertically upward direction. The third process includes irradiating the substrate with an excitation laser at a total reflection angle to generate evanescent waves, capturing a scattered light image of the particles to which an external force in the vertically downward direction has been applied, and capturing a scattered light image of the particles to which an external force in the vertically upward direction has been applied.

### [First Process]

The first process can be performed in the same manner as in the third embodiment described above.

### [Second Process]

The second process can be performed in the same manner as in step S112 and step S115 described above. That is, application of an external force in the vertically downward direction can be performed in the same manner as in step S112. Application of an external force in the vertically upward direction can be performed in the same manner as in step S115.

### [Third Process]

The third process can be performed in the same manner as in step S113, step S114, step S116, and step S117 described above. That is, capturing of a scattered light image of the particles to which an external force in the vertically downward direction has been applied can be performed in the same manner as in step S113 and step S114. Capturing of a scattered light image of the particles to which an external force in the vertically downward direction has been applied can be performed in the same manner as in step S116 and step S117.

### [Fourth Process]

The fourth process can be performed in the same manner as in step S202 in the test substance detection device of the second embodiment described above.

### [Fifth Process]

The fifth process can be performed in the same manner as in step S203 in the test substance detection device of the second embodiment described above.

### [Other Processes]

The test substance detection method of the embodiments may include other processes in addition to the first to fifth processes. Other processes include, for example, a process (sixth process) of calculating the concentration of the test substance in the sample on the basis of the number or proportion of the particles that are bound to the catcher via the test substance. The sixth process can be performed in the same manner as in step S204 described above.

### EXAMPLES

Hereinafter, the present invention will be explained using examples, but the present invention is not limited to the following examples.

### Example 1

A case in which a cover glass is used as a substrate, magnetic beads are used as particles, an antibody is used as a catcher for binding between the substrate and an antigen, an antibody is used as a labeling probe for binding between the particles and the antigen, microwells are used to hold the magnetic beads on the substrate, a magnetic force is used as an external force, and an optical microscope is used to observe particle behaviors for the purpose of detecting the antigen in a sample solution is an exemplary example.

### 1. Modification of PEG Linker to Glass Exposed Part

(1) A plurality of microwells (diameter 2.5 μm, depth 0.8 μm) made of CYTOP (registered trademark) are created on the cover glass.
(2) Silane coupling treatment is performed by immersing a glass exposed part of the cover glass in which the microwells have been prepared in an SH silane coupling agent (KBM-803, Shin-Etsu Silicone) solution.
(3) After adding 1 mg/mL biotin-PEG-maleimide cross-linker reagent/25 mM MES (2-morpholinoethanesulfonic acid) solution to the substrate prepared in (2), the substrate remains at room temperature for 1 hour.
(4) The substrate is immersed in ethanol and ultrasonic cleaning is performed for 5 minutes.
(5) The substrate is immersed in pure water and ultrasonic cleaning is performed for 5 minutes.
(6) A glass with a plurality of holes (1.2 mm diameter) for introducing solutions is prepared as a top glass and the surface is coated with CYTOP (registered trademark). A flow cell is produced by pasting this onto the microwells using a double-sided tape as a spacer.

### 2. Binding of Antibodies to PEG Linker-Modified Substrate

A streptavidin solution is injected into the microwells and remains for 30 minutes, and then the substrate is washed with PBST. Thereafter, a biotin-labeled antibody is injected into the microwells, and the antibody is immobilized on the substrate by allowing it to remain at room temperature for 1 hour.

### 3. Antibody Modification to Beads

After activating a solution of COOH-labeled magnetic beads with a diameter of 550 nm with EDC (1-(3-dimethylaminopropyl)-3-ethylcarbodiimide) and Sulfo-NHS (N-hydroxysulfosuccinimide sodium), an antibody solution is added thereto and antibodies are allowed to bind to the beads by stirring the same overnight at 4° C. Thereafter, 100 mM ethanolamine is added thereto and they are reacted for 30 minutes at room temperature.

### 4. Mixing of Sample Solution and Antibody-Labeled Magnetic Beads

The sample solution containing the antigen and the antibody-labeled magnetic beads are mixed, and stirred and mixed at room temperature for 1 hour.

5. Beads Introduction into Microwells

The mixed solution prepared in step 4 is enclosed in the flow cell. A magnetic force is applied toward the bottoms of the microwells such that the beads move rapidly into the microwells and enclosed. Immediately after enclosing, the flow cell is placed on a microscope stage.

### 6. External Force Application and Imaging

As an example, one dark-field image of the beads is captured in a state in which a magnetic field in the vertically downward direction has been applied to the flow cell on the stage. Thereafter, one dark-field image of the beads is captured along the outer periphery of the flow cell in the horizontal plane of the flow cell in a stated in which a magnetic field in each of directions of 0 degrees, 120 degrees, and 240 degrees has been applied.

### 7. Analysis

The coordinates of each bead in a state in which a magnetic force in the vertically downward direction has been applied are determined on the basis of captured images, and the moving range of each bead is estimated on the basis of based on the coordinates when a magnetic force has been applied the directions of 0 degrees, 120 degrees, and 240 degrees in the horizontal plane of the flow cell. The coordinates of the bead are determined to be the center position of the bead. As an example, the coordinates of a bead in a state in which a magnetic force in the vertically downward direction has been applied are determined to be the origin of the bead, and the sum of the absolute values of distances from the origin of the bead position when a magnetic force is applied in the respective directions in the horizontal plane is used as an index of the moving range (in addition, a method of setting a circle that passes through the coordinates when a magnetic force has been applied in directions of 0 degrees, 120 degrees, and 240 degrees and using the area of this circle as an index, and the like are conceivable. In this case, imaging in a state in which a magnetic force has been applied in the vertically downward direction is not necessary). In this case, an unbound bead is attracted to the outer periphery of the wells by the magnetic force, and thus the moving range thereof is the largest. On the other hand, a non-specifically adsorbed bead that causes multi-bond of the bead and the catching antibody on the cover glass has a narrow moving range. A bead that is bound via an antigen causes single bond between the antigen and the catching antibody and exhibits a moving range falling between an unbound bead and a non-specifically adsorbed bead. For this reason, when a histogram of the number of measured beads is created for the index of the moving range, three peaks appear in the distribution. After performing Gaussian fitting on the three peaks appearing in the distribution, beads classified within the second peak are identified as beads that are bound via the antigen (Target Positive Trap).

Beads can be identified between captured images using existing methods. In a case where microwells are used, beads contained in the same microwell can be identified as the same bead. In a case where microwells are not used, for example, coordinates in an image when a magnetic force has been applied in the vertically downward direction are used as a reference (reference coordinates), and beads present at coordinates close to the reference coordinates can be identified as the same bead in each image when a magnetic force has been applied in each direction within the horizontal plane. Since beads that are not bound to antibodies move significantly when a magnetic force has been applied in the horizontal direction, it is not possible to associate them between images. Beads other than beads associated with the reference coordinates can be determined to be unbound beads.

### Example 2

A case in which a cover glass is used as a substrate, magnetic beads carrying a fluorescent substance inside are used as particles, an antibody is used as a catcher for binding between the substrate and an antigen, an antibody is used as a labeling probe for binding between the particles and the antigen, microwells are used to hold the magnetic beads on the substrate, a magnetic force is used as an external force, and a total internal reflection fluorescence microscopy with evanescent illumination is used to observe particle behaviors for the purpose of detecting the antigen in a sample solution is an exemplary example.

### 1. Modification of PEG Linker to Glass Exposed Part

Modification of a PEG linker to a glass exposed part is performed in the same manner as in example 1.

### 2. Binding of Antibodies to PEG Linker-Modified Substrate

Binding of antibodies to a PEG linker-modified substrate is performed in the same manner as in example 1.

### 3. Antibody Labeling on Fluorescently Labeled Magnetic Beads

After activating a solution of COOH-modified fluorescently labeled magnetic beads with a diameter of 500 nm with EDC and Sulfo-NHS, an antibody solution is added thereto and the solution of the beads to which the antibody solution has been added is stirred overnight at 4° C. to cause antibodies to bind to the beads. Thereafter, 100 mM ethanolamine is added and the antibodies and the beads are reacted for 30 minutes at room temperature.

### 4. Mixing of Sample Solution and Antibody-Labeled Magnetic Beads

The sample solution containing the antigen and the antibody-labeled magnetic beads are mixed, and stirred and mixed at room temperature for 1 hour.

5. Bead Introduction into Microwells

The mixed solution prepared in step 4 is enclosed in a flow cell. Immediately after enclosing, the flow cell is placed on the microscope stage.

### 6. External Force Application and Imaging

The flow cell is placed on the stage with the side on which catching antibodies have bound facing down. In a state in which a magnetic force has been applied in the vertically downward direction from the upper side of the flow cell, an excitation laser is radiated from the bottom surface of the flow cell through an objective lens at a total reflection angle. Fluorescence generated from the beads when the beads are illuminated by evanescent waves generated on the reflective surface of the excitation laser is acquired through the objective lens. Next, in a state in which a magnetic force has been applied in the vertically upward direction from the upper side of the flow cell, an excitation laser is radiated from the bottom surface of the flow cell through the objective lens at a total reflection angle. Fluorescence generated from the beads when the beads are illuminated by evanescent waves generated on the reflective surface of the excitation laser is acquired through the objective lens.

### 7. Analysis

In two images when a magnetic force in the vertically downward direction and a magnetic force in the vertically upward direction have been applied, a difference between fluorescence brightness when a downward magnetic force has been applied and fluorescence brightness when an upward magnetic force has been applied is obtained with respect to fluorescence brightness between the same points. Since the intensity of evanescent waves is exponentially attenuated in response to the distance from the cover glass/liquid interface, the fluorescence brightness is attenuated as the beads move away from the interface. When a histogram is created with respect to the difference in fluorescence brightness, the brightness difference increases for magnetic beads that are not bound to the cover glass. On the other hand, in non-specifically adsorbed beads that are adsorbed through multi-bond via a plurality of antibodies on the cover glass, fluorescence brightness attenuation is insignificant. In the case of beads that are bound via the antigen, the beads are separated from the substrate by the distance between the antigen and antibody binding sites, and binding between the antigen and the antibody on the cover glass becomes single bond. Therefore, a bead that are bound via the antigen has a longer distance from the substrate than a bead that are bound non-specifically. As a result, for a bead that are bound via the antigen, the fluorescence brightness difference falls between those of a non-specifically adsorbed bead and an unbound bead. As a result, three peaks appear in a histogram of fluorescence brightness differences, and after performing Gaussian fitting thereon, beads classified into the second peak are identified as beads that are bound via the antigen (Target Positive Trap).

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A test substance detection device comprising:
a substrate on which a catcher having a binding ability to a test substance is immobilized;
an external force applying mechanism;
an imaging device; and
processing circuitry,
wherein the processing circuitry is configured to:
control the external force applying mechanism to apply an external force to a particle on the substrate, the particle having an ability to bind to the test substance and having undergone a reaction for binding to the test substance;
control the imaging device to image the particle on the substrate in a state in which the external force has been applied;
determine a moving range of the particle due to application of the external force on the basis of the image captured by the imaging unit; and
distinguish between (a) the particle bound to the catcher via the test substance and (b) the particle bound to the catcher without the test substance on the basis of the moving range.

2. The test substance detection device according to claim 1, wherein the catcher comprises an antibody having a binding ability to the test substance and a linker for immobilizing the antibody on the substrate.

3. The test substance detection device according to claim 1, wherein the particle is a particle on which an antibody having a binding ability to the test substance is immobilized.

4. The test substance detection device according to claim 1, wherein the processing circuitry controls the external force applying mechanism to apply an external force in at least one direction within a horizontal plane to the particle on the substrate.

5. The test substance detection device according to claim 1, wherein the processing circuitry controls the external force applying mechanism to apply an external force in a vertically downward direction and an external force in a vertically upward direction to the particle on the substrate.

6. The test substance detection device according to claim 5, further comprising a light source that irradiates the substrate with an excitation laser at a total reflection angle.

7. A test substance detection method comprising:
(1) reacting a test substance with a particle having a binding ability to the test substance on a substrate on which a catcher having a binding ability to the test substance are immobilized;
(2) applying an external force to the particle on the substrate after (1);
(3) imaging the particle on the substrate in a state in which the external force has been applied;
(4) determining a moving range of the particle due to application of the external force on the basis of the captured image; and
(5) distinguishing between (a) the particle binding to the catcher via the test substance and (b) the particle binding to the catcher without the test substance on the basis of the moving range.

8. The test substance detection method according to claim 7, wherein the catcher comprises an antibody having a binding ability to the test substance and a linker for immobilizing the antibodies on the substrate.

9. The test substance detection method according to claim 7, wherein the particle is particle on which an antibody having a binding ability to the test substance is immobilized.

10. The test substance detection method according to claim 7, wherein (2) comprises applying an external force in at least one direction within a horizontal plane to the particle on the substrate, and
(3) comprises imaging the particle to which an external force in one direction within the horizontal plane has been applied.

11. The test substance detection method according to claim 7, wherein (2) comprises applying an external force in a vertically downward direction and applying an external force in a vertically upward direction to the particle on the substrate, and
(3) comprises irradiating the substrate with an excitation laser at a total reflection angle to generate evanescent waves, capturing a scattered light image of the particle to which the external force in the vertically downward direction has been applied, and capturing a scattered light image of the particle to which the external force in the vertically upward direction has been applied.
